(19) United States
(12) Patent Application Publication
(10) Pub. No.: US 2010/0173371 A1
(43) Pub. Date: Jul. 8, 2010

(54) Medium Comprising Fermented Korean Hot Pepper Paste Or Soybean Sauce And Production Method Of Gamma - Aminobutyric Acid

(75) Inventors: Myoung-hee Jeon, Daejeon (KR); Seung-jin Lee, Choongcheongnam-do (KR); Byoung-koo Kwon, Choongcheongnam-do (KR); Yeong-il Chang, Daejeon (KR); Hee-kyoung Park, Seoul (KR); Jun-bong Choi, Seoul (KR)

Correspondence Address: SWANSON & BRATSCHUN, L.L.C., 8210 SOUTHPARK TERRACE, LITTLETON, CO, 80120, US

(73) Assignee: CJ CHEILJEDANG CORPORATION, Seoul (KR)

(21) Appl. No.: 12/095,552

(22) PCT Filed: Dec. 22, 2006
(86) PCT No.: PCT/KR2006/005662

§ 371 Date: Jul. 31, 2008

(30) Foreign Application Priority Data
Dec. 27, 2005 (KR) 10-2005-0130762

(51) Int. Cl. C12P 7/42; C12N 1/20

(52) U.S. Cl. 435/146; 435/252.9

(57) ABSTRACT

The present invention relates to a medium composition comprising fermented Korean hot pepper paste, undiluted solution of brewed soy sauce or acid hydrolyzed soy sauce, and a method for producing gamma-aminobutyric acid (GABA) using the medium. More precisely, the present invention relates to a medium composition for culturing lactobacillus having GAD activity, the composition comprising fermented hot pepper paste, brewed soy sauce or acid hydrolyzed soy sauce, and a method for producing a high concentration of gamma-aminobutyric acid from the medium additionally added with glutamic acid or glutamate. The present invention provides a method of producing a high concentration of gamma-aminobutyric acid using the above medium with low production costs. Since the by-products of pastes are used as medium compositions, this medium itself can be added to other pastes or foods to produce functional foods containing gamma-aminobutyric acid.

### TECHNICAL FIELD

The present invention relates to a medium composition comprising fermented Korean hot pepper paste, undiluted solution of brewed soy sauce or acid hydrolyzed soy sauce, and a method of preparing gamma-aminobutyric acid. More particularly, the present invention relates to a medium composition for culturing lactobacillus having glutamate decarboxylase activity which comprising fermented Korean hot pepper paste, undiluted solution of brewed soy sauce or acid hydrolyzed soy sauce, and a method of preparing highly concentrated gamma-aminobutyric acid using the medium additionally added with glutamic acid or glutamate.

### BACKGROUND ART

Gamma-aminobutyric acid (GABA) is a non-protein constituent amino acid which has molecular weight of 103.12 and melting point of 203° C. This amino acid is thermo-stable and also highly soluble in water.

The GABA production mechanism has been more precisely revealed in microorganisms than in animals or plants. During the proliferation of a microorganism, excessive extracellular metabolites are accumulated in the late stage of proliferation, by which the balance between extracellular and intracellular hydrogen ions (H⁺) is broken; to regulate such an imbalance, GABA is generated. That is, when extracellular glutamate transfers inside cells, the carboxyl group of the glutamate is substituted with the intracellular H⁺, so that intracellular H⁺ is consumed to result in generation of CO₂, during which GABA is generated. The GAD enzyme associated with this GABA generation is expressed and activated to maintain a constant pH so as to resist acid stress. The optimum pH differs between microorganisms, but is generally in the range of 4.2˜4.7. 5′-pyridoxal phosphate (PLP) is an example of a coenzyme.

GABA, found in animal brains, is a neurotransmission inhibitor which plays an important role in the central nervous system. GABA has been recognized as having preventive and/or therapeutic effects on paralysis, dementia, mental concentration enhancing and memory enhancing and insomnia by enhancing brain cell metabolism.

According to a recent research on the effects of GABA in Japan, embryo bud of rice accumulating GABA was orally administered to aged people suffering from menopausal disorder and/or mental disorder at a dose of 26.5 [symbol] of GABA per day. As a result, according to the above report, mental disorders such as headache and depression or various symptoms of menopausal disorder were improved by approximate 75%.

Another important activity of GABA accelerates sodium ion discharge through urine, so it is known to lower blood pressure to hypertension which may be caused from excessive intake of salt.

Another study related to GABA reported that the level of GABA in the blood of an alcoholic was significantly lower than that of a normal person, so the study suggested that GABA accelerates alcohol metabolism. In addition, GABA is known to be involved in the regulation of growth hormone secretion, diuretic action, preventing obesity, relieving pain and tension, stress control and activation of liver functions, etc, so GABA has become a pharmacologically interesting compound based on such various physiological effects.

GABA is recognized not only as a medicinal compound having various functions and effects but also as a functional food material. As a medicinal compound, GABA has been applied by intravenous injection, which is administered to treat stroke, head trauma and sequelae of cerebral aneurysm to increase cerebral blood flow and cerebral metabolism. As a functional food, GABA has been applied at high concentration to green tea, which is called GABARON TEA in Japan, and to beverages and soybean paste (miso) as well.

GABA is included in foods such as vegetables, fruits, and grains, but the concentrations therein are very low and thus any beneficial effect is in doubt. So, various attempts to produce highly concentrated GABA have been made or are being made.

Methods to increase GABA concentration has mainly been tried in plants, the examples of which are cold shock, mechanism stimulation, heat shock, hypoxia, cytosolic acidification, water stress, phytohormones, etc caused from external environmental factors. Recently, with the advance of molecular biological techniques, increased GABA concentration has tried to be achieved by introducing GAD or calmodulin gene into tobacco plants. However, the above methods need physical installations and professional knowledge.

As an alternative, GABA production is being tried using microorganisms having GAD activity such as mold, E. coli and lactobacillus. In particular, lactobacillus is preferably used to produce GABA as a food additive, since lactobacillus has a wide application range and originates from generally consumed fermented foods. However, lactobacillus is a strain requiring various nutrients, so the culturing therefor is troublesome and the price of the culture medium is expensive. Even though there are differences according to the species, lactobacillus generally needs various amino acids, vitamins, salts and specific peptides. If there is any deficiency in necessary nutrition for a specific strain, the strain will not grow properly, resulting in a poor yield of fermented products.

### DISCLOSURE OF INVENTION

### Technical Problem

It is an object of the present invention, in order to overcome the problems of the conventional method, to provide an optimum medium composition for the culture of lactobacillus having glutamate decarboxylase (GAD) activity, which comprising fermented Korean hot pepper paste, undiluted solution of brewed soy sauce or acid hydrolyzed soy sauce in order to mass-produce gamma-aminobutyric acid (GABA).

It is another object of the present invention to provide a method of producing gamma-aminobutyric acid (GABA) at high concentration by culturing lactobacillus having glutamate decarboxylase (GAD) activity in the optimum medium for lactobacillus culture.

### Technical Solution

To achieve the above objects, the present invention provides a medium composition for lactobacillus culture which comprising fermented Korean hot pepper paste, undiluted solution of brewed soy sauce or acid hydrolyzed soy sauce to produce gamma-aminobutyric acid.

The medium composition of the present invention can further comprises glutamic acid or glutamate.

The fermented Korean hot pepper paste may be a fermented product originating from one or more materials selected from a group consisting of wheat, wheat bran, wheat corn, rice, barley, sorghum, corn, oat, buck wheat, millet or their processed products.

The undiluted solution of brewed soy sauce or acid hydrolyzed soy sauce may be those originating from one or more materials selected from a group consisting of soybeans, bean flour, defatted soybean flour, kidney beans, mung beans or their processed products.

The medium composition can further comprises one or more materials selected from a group consisting of glucose, NaCl, glutamate, pyridoxal phosphate, garlic, and tomato puree.

The present invention also provides a method of producing gamma-aminobutyric acid in the culture solution after culturing lactobacillus in the medium comprising fermented Korean hot pepper paste, undiluted solution of brewed soy sauce or acid hydrolyzed soy sauce.

The present invention also provides a method of producing gamma-aminobutyric acid further comprising the step of adding glutamic acid or glutamate during the culture.

The present invention also provides a method of producing gamma-aminobutyric acid, wherein the fermented Korean hot pepper paste is a fermented product originating from one or more materials selected from a group consisting of wheat, wheat bran, wheat corn, rice, barley, sorghum, corn, oat, buck wheat, millet and their processed products.

The present invention also provides a method of producing gamma-aminobutyric acid, wherein the undiluted solution of brewed soy sauce and acid hydrolyzed soy sauce originating from one or more materials selected from a group consisting of soybeans, bean flour, defatted soybean flour, kidney beans, mung beans and their processed products.

The present invention also provides a method of producing gamma-aminobutyric acid, wherein the medium further comprises one or more materials selected from a group consisting of sugar, NaCl, glutamic acid, pyridoxal phosphate, garlic and tomato puree.

According to the conventional method of producing GABA based on lactobacillus culture, glutamate is added to a mixed medium (MRS, LBS, skim milk, tomato juice, etc), and the medium is sterilized, cooled down and inoculated with lactobacillus for culture. However, the high medium costs increase the GABA production costs, which is not suitable for industrial application.

In the course of research on how to produce GABA by culturing lactobacillus having GAD activity and how to test the possibility of replacing the high cost MRS medium with a less expensive medium to overcome the disadvantages of the conventional method in industrial application, and how to establish a fermentation method for the mass-production of GABA, the present inventors found out the fact that lactobacillus can grows and produces GABA in a mixture of fermented Korean hot pepper paste and undiluted solution of brewed soy sauce. Therefore, the present inventors completed this invention by optimizing the conditions for GAD generation and activation, and confirmed that GABA can be mass-produced by fed-batch culture with repeated supply of a substrate during the culture.

Hereinafter, the present invention is described in detail.

The present invention provides a medium composition for lactobacillus culture which comprises fermented Korean hot pepper paste, undiluted solution of brewed soy sauce or acid hydrolyzed soy sauce to produce gamma-aminobutyric acid.

The present invention also provides a method of producing gamma-aminobutyric acid from the culture solution after culturing lactobacillus in the medium comprising fermented Korean hot pepper paste, undiluted solution of brewed soy sauce or acid hydrolyzed soy sauce.

The concentration of amino-nitrogen in the fermented Korean hot pepper paste, undiluted solution of brewed soy sauce or acid hydrolyzed soy sauce is preferably, 10˜500 [symbol] % and is more preferably, 10˜300 [symbol] %.

The fermented Korean hot pepper paste may originate from one or more materials selected from a group consisting of wheat, wheat bran, wheat corn, rice, barley, sorghum, corn, oat, buck wheat, millet and their processed products.

The present invention also provides a method of producing gamma-aminobutyric acid wherein the undiluted solution of brewed soy sauce and acid hydrolyzed soy sauce may originate from one or more materials selected from a group consisting of soybeans, bean flour, defatted soybean flour, kidney beans, mung beans and their processed products.

The medium composition can further comprises one or more materials selected from a group consisting of glucose, NaCl, glutamate, pyridoxal phosphate (PLP), garlic and tomato puree.

The added glucose may include any carbon source that is able to be fermented by lactobacillus at addition of glucose; the preferable concentration is 0˜10 w/w % and more preferably 0˜5 w/w % by weight.

The preferable concentration of added NaCl is 0˜5.0 w/w % and 0˜3 w/w % is more preferred.

The preferable concentration of added PLP is 0.1 μmol˜100 m [symbol] and 0.1 μmol˜10 m [symbol] is more preferred.

The preferable concentration of added phosphate is 0.1-5.0 w/w % and 0.1-3 w/w % is more preferred.

The added garlic may be both raw garlic and processed garlic products; the preferable concentration is 0.5˜10 w/w % and 0.5˜5 w/w % is more preferred.

The tomato puree may be not only tomato puree but also fresh tomato and processed tomato products; the preferable concentration is 0.5˜10 w/w % and 0.5˜5 w/w % is more preferred.

The lactobacillus added for the culture may be any strain as long as it has strong GAD activity and is sitologically acceptable. For example, one or more strains selected from a group consisting of Lactobacillus brevis, Lactobacillus sakei, Lactobacillus acidophillus, Leuconostoc plantarum, Leuconostoc mesentroides, Bifidobacterium breve, Bifidobacterium longum, Bifidobacterium infantis, Bifidobacterium thermophyllum, Streptococcus faecalis and Streptococcus thermophillus can be used.

It is preferable not to add salts considering the growth conditions of lactobacillus, but the fermentation products used as a medium may contain salts. If the concentration of salts is less than 4 w/w %, it will not affect the generation of GABA.

The amount of lactobacillus added for the fermentation is determined by the strain, and generally 10⁶˜10⁷ CFU/ [symbol] is preferred. If the amount of lactobacillus added is less than 10⁶ CFU/ [symbol] , the duration of culture will be extended. On the contrary, if the amount is more than 10⁷ CFU/ [symbol] , economical efficiency will be reduced in addition to decrease in the enzyme activity.

The preferable time point of adding lactobacillus to the medium is when the medium is sterilized and cooled down to around 30° C. so as not to inhibit the growth of lactobacillus.

Glutamate is dissolved in the medium, which is sterilized, cooled down, and added to the medium several times at an interval of 4˜6 hours at a concentration of 10˜10,000 [symbol] % to the total volume of the medium.

To induce maximum GAD activity in fed-batch culture, the pH is preferably 4.2˜5.0 and it can be adjusted by adding an acid to the culture solution at an interval of 5˜6 hours.

The acid added for adjusting the pH herein can be used either an inorganic acid or an organic acid.

The inorganic acid may be one or more compounds selected from a group consisting of sulfuric acid, hydrochloric acid, phosphoric acid and nitric acid. The organic acid may be one or more compounds selected from a group consisting of lactic acid, acetic acid, malic acid, citric acid and formic acid.

GABA was measured by gas chromatography (GC) with the unit of 0%. The conversion rate (%) was calculated by the following formula: (GABA mmol/added glutamate mmole)×100.

In a preferred embodiment of the present invention, grain was mixed with wheat flour koji and was then aged. The resulting fermented product (hereinafter referred to as the “fermented Korean hot pepper paste”) and soy koji were soaked in salt water. After aging, the fermented solution (hereinafter referred to as the “undiluted solution of brewed soy sauce”) was separated, to which phosphoric acid, PLP (5′-pyridoxal phosphate), garlic, and tomato puree were added respectively as nutrition sources. Then, to prepare the medium of the present invention, glutamic acid was added thereto. The medium was inoculated with Lactobacillus brevis, followed by culture for 26 hours. The pH of the medium was adjusted to 4.7, and 3,000 [symbol] % of glutamic acid was added several times at intervals of 6 hours during the 50 hour culture period to produce a high concentration of GABA.

The present invention suggests that a medium comprising fermented hot pepper paste and undiluted solution of brewed soy sauce, together with a small amount of PLP, phosphoric acid, garlic or tomato puree, can provide optimum conditions for the growth of lactobacillus, so that this medium may be an excellent industrial medium that will take the place of the conventional MRS medium for producing GABA. The present invention further suggests that GABA is mass-produced with low expense by fed-batch culture, in which the pH of the medium is adjusted to maintain GAD secretion and maximum activity of GABA, and glutamate is repeatedly added with intervals so as not to inhibit the substrate.

### BRIEF DESCRIPTION OF THE DRAWINGS

FIG. 1 is a flow diagram illustrating the processes of preparing an optimum medium and fed-batch culture for the production of gamma-aminobutyric acid at high concentration.

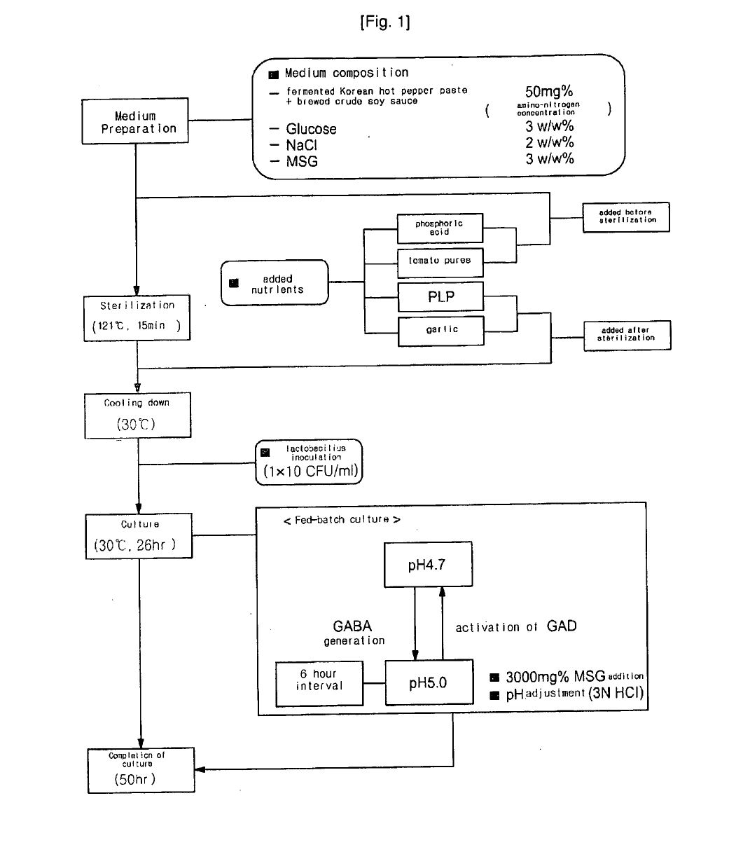

### BEST MODE FOR CARRYING OUT THE INVENTION

Hereinafter, the embodiments of the present invention will be described in detail with reference to the accompanying drawings. The invention is only exemplified by the following examples for understanding, therefore which should not be understood to limit the scope of the present invention.

### Example 1

To investigate the optimum substrate concentration, glutamate (Monosodium Glutamate: MSG) was added with 1 w/w % to the total weight of MRS medium (Peptone 10 g, Beef extract 10 g, Yeast extract 5 g, Dextrose 20 g, Polysorbate 80 1 g, Ammonium citric acid 2 g, Sodium acetic acid 5 g, Magnesium sulfate 0.1 g, Manganese sulfate 0.05 g, Dipotassium phosphate 2 g, Distilled water 1,000 [symbol] , pH 6.5), followed by sterilization at 121° C. for 15 minutes. After cooling down, Lactobacillus brevis was inoculated into the medium at the concentration of 1×10⁷ CFU/ [symbol] , followed by culture for 26 hours. GABA was measured and the result is shown in Table 1.

### Example 2

An experiment was performed in the same manner as described in Example 1, except that glutamate (MSG) was added with 2 w/w % to the total weight. GABA was measured and the result is shown in Table 1.

### Example 3

An experiment was performed in the same manner as described in Example 1, except that glutamate (MSG) was added with 3 w/w % to the total weight. GABA was measured and the result is shown in Table 1.

### Example 4

An experiment was performed in the same manner as described in Example 1, except that glutamate (MSG) was added with 4 w/w % to the total weight. GABA was measured and the result is shown in Table 1.

### Example 5

An experiment was performed in the same manner as described in Example 1, except that glutamate (MSG) was added with 5 w/w % to the total weight. GABA was measured and the result is shown in Table 1.

### Example 6

An experiment was performed in the same manner as described in Example 1, except that the amino-nitrogen concentration for fermented hot pepper paste (wheat flour koji and grain were mixed, aged and fermented) was 30 [symbol] % to the total weight of medium, glucose and NaCl were added to make the concentration of sugar and salts therein become 3 w/w % and 2 w/w % respectively, and glutamate (MSG) was added with 3 w/w % to the medium. GABA was measured and the result is shown in Table 1.

### Example 7

An experiment was performed in the same manner as described in Example 6, except that fermented hot pepper paste and undiluted solution of brewed soy sauce were mixed so that the amino-nitrogen concentrations for the fermented hot pepper paste and the brewed soy sauce (bean koji was soaked in salt water, aged and separated) respectively become 30 mg % and 20 [symbol] % to total weight of medium. GABA level was measured and the result is shown in Table 1.

### Example 7-1

An experiment was performed in the same manner as described in Example 6, except that fermented hot pepper paste and undiluted solution of brewed soy sauce were mixed so that the amino-nitrogen concentrations for the fermented hot pepper paste and the acid hydrolyzed soy sauce (protein or carbohydrate containing material was hydrolyzed with acid and the filtrate was processed) respectively become 30 mg % and 20 [symbol] % to total weight of medium. GABA level was measured and the result is shown in Table 1.

### Example 8

An experiment was performed in the same manner as described in Example 6, except that fermented hot pepper paste and undiluted solution of brewed soy sauce were mixed so that the amino-nitrogen concentrations for the fermented hot pepper paste and the brewed soy sauce respectively become 30 mg % and 40 [symbol] % to total weight of medium. GABA level was measured and the result is shown in Table 1.

### Example 8-1

An experiment was performed in the same manner as described in Example 6, except that fermented hot pepper paste and undiluted solution of brewed soy sauce were mixed so that the amino-nitrogen concentrations for the fermented hot pepper paste and the acid hydrolyzed soy sauce respectively become 30 mg % and 40 [symbol] % to total weight of medium. GABA level was measured and the result is shown in Table 1.

### Example 9

An experiment was performed in the same manner as described in Example 7, except that 0.1 μmol of 5′-pyridoxal phosphate (PLP) to the total volume was added after the medium had been sterilized. GABA level was measured and the result is shown in Table 1.

### Example 10

An experiment was performed in the same manner as described in Example 7, except that 1.0 μmol of 5′-pyridoxal phosphate (PLP) to the total volume was added after the medium had been sterilized for total weight. GABA level was measured and the result is shown in Table 1.

### Example 11

An experiment was performed in the same manner as described in Example 7, except that 10 μmol of 5′-pyridoxal phosphate (PLP) to the total volume was added after the medium had been sterilized for total weight. GABA level was measured and the result is shown in Table 1.

### Example 12

An experiment was performed in the same manner as described in Example 7, except that 0.1% w/w of phosphoric acid to the total weight was added. GABA level was measured and the result is shown in Table 1.

### Example 13

An experiment was performed in the same manner as described in Example 7, except that 0.2% w/w of phosphoric acid to the total weight was added. GABA level was measured and the result is shown in Table 1.

### Example 14

An experiment was performed in the same manner as described in Example 7, except that 0.3% w/w of phosphoric acid to the total weight was added. GABA level was measured and the result is shown in Table 1.

### Example 15

An experiment was performed in the same manner as described in Example 7, except that 1.0% w/w of garlic to the total weight was added after the medium had been sterilized. GABA level was measured and the result is shown in Table 1.

### Example 16

An experiment was performed in the same manner as described in Example 7, except that 2.0% w/w of garlic to the total weight was added after the medium had been sterilized. GABA level was measured and the result is shown in Table 1.

### Example 17

An experiment was performed in the same manner as described in Example 7, except that 3.0% w/w of garlic to the total weight was added after the medium had been sterilized. GABA level was measured and the result is shown in Table 1.

### Example 18

An experiment was performed in the same manner as described in Example 7, except that 0.5% w/w of tomato puree to the total weight was added. GABA level was measured and the result is shown in Table 1.

### Example 19

An experiment was performed in the same manner as described in Example 7, except that 1.0% w/w of tomato puree to the total weight was added. GABA level was measured and the result is shown in Table 1.

### Example 20

An experiment was performed in the same manner as described in Example 7, except that 1.5% w/w of tomato puree to the total weight was added. GABA level was measured and the result is shown in Table 1.

### Example 21

An experiment was performed in the same manner as described in Example 12, except that a fermentor was used to produce a high concentration of GABA. GABA level was measured and the result is shown in Table 2.

### Example 22

An experiment was performed in the same manner as described in Example 1, except that glutamate was added at 3,000 mg % to the total weight of medium to the fermentor after culturing for 26 hours while the pH was adjusted to 4.7 by adding 3 N HCl, followed by further culture for 6 hours (total 32 hours). GABA level was measured and the result is shown in Table 2.

### Example 23

An experiment was performed in the same manner as described in Example 2, except that the culture time was 38 hours in total. GABA level was measured and the result is shown in Table 2.

### Example 24

An experiment was performed in the same manner as described in Example 2, except that the culture time was 44 hours in total. GABA level was measured and the result is shown in Table 2.

### Example 25

An experiment was performed in the same manner as described in Example 2, except that the culture time was 50 hours in total. GABA level was measured and the result is shown in Table 2.

TABLE 1

| | GABA | Conversion |
|---|---|---|
| Example | (mg %) | rate (%) |
| 1 | 550 | 100 |
| 2 | 1,100 | 100 |
| 3 | 1,651 | 100 |
| 4 | 1,613 | 73 |
| 5 | 1,632 | 59 |
| 6 | 1,074 | 65 |
| 7 | 1,322 | 80 |
| 7-1 | 1,302 | 79 |
| 8 | 1,289 | 78 |
| 8-1 | 1,278 | 77 |
| 9 | 1,570 | 95 |
| 10 | 1,652 | 100 |
| 11 | 1,652 | 100 |
| 12 | 1,652 | 100 |
| 13 | 1,619 | 98 |
| 14 | 1,553 | 94 |
| 15 | 1,652 | 100 |
| 16 | 1,553 | 94 |
| 17 | 1,504 | 91 |
| 18 | 1,652 | 100 |
| 19 | 1,619 | 98 |
| 20 | 1,570 | 95 |

From the results of the above preferred embodiments investigating the substrate inhibiting concentration in MRS medium which is known as an optimum medium for lactobacillus growth, it was confirmed that the preferred concentration of glutamate so as not to inhibit the substrate was 3 w/w %, as shown in Example 3.

GABA conversion rate was the highest in Example 7 in which the fermented Korean hot pepper paste and the brewed soy sauce were used to provide a proper concentration and mixture ratio of amino-nitrogen, and GABA conversion rate was also high in Example 7-1 in which the fermented Korean hot pepper paste and the acid hydrolyzed soy sauce were used.

When the medium of Example 7 was supplemented with 1.0 μmol of PLP, 0.1 w/w % of phosphoric acid, 1.0 w/w % of garlic, and 0.5 w/w % pf tomato puree, the conversion rate reached 100% as shown in Examples 9, 12, 15 and 18.

When the medium was supplemented with the fermented Korean hot pepper paste, the brewed soy sauce or acid hydrolyzed soy sauce alone, there appeared to be inadequate essential nutrients for the growth of lactobacillus. Added PLP was involved in activation of GAD as a coenzyme and phosphoric acid was involved in an essential nutrition factor for the growth of lactobacillus. Garlic and tomato puree supplemented the nutrients that the fermented hot pepper paste and the soy sauce lacked. Therefore, all these materials together contributed to the increase of conversion rate.

Therefore, the present invention found out the fact that the optimum medium for Lactobacillus suitable for producing GABA can be prepared by adding small amount of PLP or phosphoric acid or tomato puree in the mixture of the fermented Korean hot pepper paste and the brewed soy sauce as alternative industrial medium to replace the conventional MRS medium.

TABLE 2

| | | | Converted | | |
|---|---|---|---|---|---|
| | | | amount of | | |
| | | Amount of | glutamic | Amount of | |
| | Culture | glutamate | acid | GABA | Conversion |
| Example | time | (mg %) | (mmole) | (mmole) | Rate (%) |
| 21 | 0-26 | 3,000 | 160 | | 100 |
| | | (3,000)* | (160)** | 160 | |
| 22 | 26-32 | 3,000 | 160 | | 100 |
| | | (6,000)* | (320)** | 321 | |
| 23 | 32-38 | 3,000 | 160 | | 100 |
| | | (9,000)* | (480)** | 481 | |
| 24 | 38-44 | 3,000 | 160 | | 98 |
| | | (12,000)* | (640)** | 629 | |
| 25 | 44-50 | 3,000 | 160 | | 96 |
| | | (15,000)* | (800)** | 770 | |

( )* accumulated amount of added glutamate
( )** accumulated amount of glutamic acid

As indicated in the above results, after 26 hours of primary culture, the pH was adjusted to 4.7 and 3,000 [symbol] % of glutamate (MSG) was added with intervals of 6 hours, by which the conversion rate reached 100%. However, as shown in the results of Example 24 and Example 25, the conversion rate gradually decreased, which is believed to be because the microorganisms therein were about to die and thus their activities were significantly reduced.

As shown in Example 25, the final GABA concentration produced after 50 hours of culture was 770 mmole.

### INDUSTRIAL APPLICABILITY

As explained above, according to the method of the present invention, lactobacillus generating gamma-aminobutyric acid is cultured on a medium composition supplemented with fermented Korean hot pepper paste, undiluted solution of brewed soy sauce or acid hydrolyzed soy sauce, which facilitates the production of a high concentration of gamma-aminobutyric acid within a short period of time with less expense.

The materials added to the medium composition of the present invention are all used for producing traditional pastes, so that the medium itself can be directly applied to the production of pastes and seasoning foods or added to functional foods and medicinal drugs directly or after being purified as gamma-aminobutyric acid.

1. A medium composition for culturing lactobacillus to produce gamma-aminobutyric acid (GABA), the composition comprising fermented Korean hot pepper paste, undiluted solution of brewed soy sauce or acid hydrolyzed soy sauce.

2. The medium composition according to claim 1,
wherein the medium composition further comprises glutamic acid or glutamate.

3. The medium composition according to claim 1,
wherein the fermented Korean hot pepper paste originates from one or more materials selected from a group consisting of wheat, wheat bran, wheat corn, rice, barley, sorghum, corn, oat, buck wheat, millet and their processed products.

4. The medium composition according to claim 1,
wherein the brewed soy sauce and acid hydrolyzed soy sauce originates from one or more materials selected from a group consisting of soybeans, bean flour, defatted soybean flour, kidney beans, mung beans and their processed products.

5. The medium composition according to claim 1,
wherein the medium composition further comprises one or more materials selected from a group consisting of glucose, NaCl, glutamate, pyridoxal phosphate, garlic and tomato puree.

6. A method of producing gamma-aminobutyric acid, comprising the steps of culturing lactobacillus for producing gamma-aminobutyric acid in a medium comprising fermented Korean hot pepper paste, undiluted solution of brewed soy sauce or acid hydrolyzed soy sauce, and isolating gamma-aminobutyric acid from the culture solution.

7. The method of producing gamma-aminobutyric acid according to claim 6,
wherein glutamic acid or glutamate is additionally added to the medium during the culturing.

8. The method of producing gamma-aminobutyric acid according to claim 6,
wherein the fermented Korean hot pepper paste originates from one or more materials selected from a group consisting of wheat, wheat bran, wheat corn, rice, barley, sorghum, corn, oat, buck wheat, millet and their processed products.

9. The method of producing gamma-aminobutyric acid according to claim 6,
wherein the brewed soy sauce and acid hydrolyzed soy sauce originate from one or more materials selected from a group consisting of soybeans, bean flour, defatted soybean flour, kidney beans, mung beans and their processed products.

10. The method of producing gamma-aminobutyric acid according to claim 6,
wherein the medium is further comprising one or more materials selected from a group consisting of glucose, NaCl, glutamate, pyridoxal phosphate, garlic and tomato puree.
